(19) United States
(12) Patent Application Publication
(10) Pub. No.: US 2026/0354786 A1
(43) Pub. Date: Nov. 12, 2026

(54) ULTRASONIC WAVE-BASED AUTOMATED BLOOD PRESSURE MONITORING METHOD AND SYSTEM

(71) Applicant: BRATTEA MEDTECH CO., LTD., Shanghai (CN)

(72) Inventors: Jiulin GUO, Shanghai (CN); Hanli HUANG, Shanghai (CN); Yan WANG, Shanghai (CN)

(73) Assignee: BRATTEA MEDTECH CO., LTD., Shanghai, SH (CN)

(21) Appl. No.: 19/715,806

(22) Filed: Jun. 22, 2026

Related U.S. Application Data
(63) Continuation of application No. PCT/CN2024/144701, filed on Dec. 31, 2024.

(30) Foreign Application Priority Data
Dec. 22, 2023 (CN) 202311792009.6

(51) Int. Cl. A61B 8/04; A61B 8/00; A61B 8/06; G01S 15/58

(52) U.S. Cl. CPC A61B 8/04; A61B 8/06; A61B 8/4477; A61B 8/4483; A61B 8/461; A61B 8/488; A61B 8/58; G01S 15/582

(57) ABSTRACT

Disclosed are an ultrasonic wave-based automated blood pressure monitoring method and system. The method comprises: transmitting an ultrasonic wave based on a flexible ultrasonic transducer array provided on a body surface of a user, and receiving a vascular image of a target region to identify an arterial blood vessel; determining a working region of the flexible ultrasonic transducer array based on the position of the arterial blood vessel, the working region comprising a first ultrasonic transducer for measuring a blood vessel diameter and a second ultrasonic transducer for measuring a blood flow; measuring the blood vessel diameter and the blood flow using the first and second ultrasonic transducers; calculating a current blood pressure of the user through a preset algorithm based on the blood vessel diameter and the blood flow; and performing next blood pressure monitoring after a preset interval.

### BACKGROUND

### Technical Field

The present invention relates to an ultrasonic wave-based automatic blood pressure monitoring method, and also relates to a corresponding automatic blood pressure monitoring system, which belongs to the technical field of medical instruments.

### Related Art

A blood pressure plays an important role in clinical medical diagnosis, prevention, and treatment strategies, as the blood pressure can accurately reflect specific cardiovascular information of a patient, and may be used by a physician to perform a subsequent corresponding diagnostic solution. Blood pressure measurement methods are classified into an invasive blood pressure measurement method and a non-invasive blood pressure measurement method. As a “gold standard” for blood pressure measurement, the invasive blood pressure measurement method offers an incontrovertible accuracy, but has a problem of complex operations. Currently, the non-invasive blood pressure measurement method used in clinical practice generally includes arterial tonometry, an arterial volume clamp method, a pulse wave measurement method, and an ultrasonic blood pressure measurement method that is in a research and development stage. A sensor used in the arterial tonometry features relatively high sensitivity and relatively high measurement accuracy. However, a difficulty exists in fixing a measurement position of the sensor for a long time. The arterial volume clamp method provides high measurement accuracy, but cannot satisfy a requirement of the patient for comfort. The pulse wave measurement method does not need an inflatable cuff, thereby achieving comfort, but the measurement accuracy still needs to be improved. The ultrasonic blood pressure measurement method combines advantages of both comfort and high accuracy.

However, the existing ultrasonic blood pressure measurement method has the following problems: the sensor has an excessively large volume, a quantity of sensors is small, and a position of the sensor is prone to deviate. Therefore, a more comfortable, convenient, and accurate method for 24-hour continuous blood pressure monitoring is urgently needed.

### SUMMARY

A primary technical problem to be resolved in the present invention is to provide an ultrasonic wave-based automatic blood pressure monitoring method.

Another technical problem to be resolved in the present invention is to provide an ultrasonic wave-based automatic blood pressure monitoring system.

To achieve the foregoing technical objective, the present invention adopts the following technical solutions.

According to a first aspect of embodiments of the present invention, an ultrasonic wave-based automatic blood pressure monitoring method is provided, including the following steps:
transmitting an ultrasonic wave based on a flexible ultrasonic transducer array arranged on a body surface of a user, and receiving a vascular image of a target region, to identify an arterial blood vessel;
determining a working region of the flexible ultrasonic transducer array based on a position of the arterial blood vessel, where the working region includes a first ultrasonic transducer configured to measure a blood vessel diameter, and a second ultrasonic transducer configured to measure a blood flow rate and a blood flow direction;
measuring a blood vessel diameter of the arterial blood vessel by using the first ultrasonic transducer in the working region;
measuring a blood flow rate and a blood flow direction of the arterial blood vessel by using the second ultrasonic transducer in the working region;
calculating a current blood pressure of the user through a preset algorithm based on the blood vessel diameter and the blood flow rate of the arterial blood vessel, to complete current blood pressure monitoring; and
performing next blood pressure monitoring at a preset interval.

Preferably, the calculating a current blood pressure of the user through a preset algorithm and based on the blood vessel diameter and the blood flow rate of the arterial blood vessel is replaced with:
repeatedly calibrating blood pressures of the user a plurality of times, to obtain calibrated blood pressure values of the user;
performing parameter fitting on the blood vessel diameter and the blood flow rate of the arterial blood vessel by using the calibrated blood pressure values of the user; and
calculating the current blood pressure of the user through the preset algorithm based on the blood vessel diameter and the blood flow rate after the parameter fitting.

Preferably, the flexible ultrasonic transducer array includes a plurality of rows of first ultrasonic transducers arranged along a first direction and a plurality of rows of second ultrasonic transducers arranged along a second direction, the plurality of rows of first ultrasonic transducers and the plurality of rows of second ultrasonic transducers are arranged in parallel to each other, and two rows of second ultrasonic transducers are symmetrically arranged on two sides of each row of first ultrasonic transducers, where
when the flexible ultrasonic transducer array is parallel to a skin surface of the user, the first direction is perpendicular to the skin surface of the user, and the second direction is inclined to the skin surface of the user.

Preferably, before the calculating a current blood pressure of the user through a preset algorithm, the method further includes:
obtaining a relative direction between the first ultrasonic transducer in the working region and the arterial blood vessel based on the position of the arterial blood vessel;
obtaining a relative angle between the second ultrasonic transducer in the working region and the arterial blood vessel based on the position of the arterial blood vessel;
correcting the blood vessel diameter based on the relative direction, to obtain the corrected blood vessel diameter;
correcting the blood flow rate based on the relative angle, to obtain the corrected blood flow rate; and
calculating the current blood pressure of the user through the preset algorithm based on the corrected blood vessel diameter and the corrected blood flow rate.

Preferably, the transmitting an ultrasonic wave based on a flexible ultrasonic transducer array arranged on a body surface of a user, and receiving a vascular image of a target region, to identify an arterial blood vessel specifically includes:
transmitting ultrasonic waves toward the target region of the user along the first direction through the plurality of rows of first ultrasonic transducers, to calculate a blood vessel diameter along the first direction through two spaced echo signals;
obtaining a waveform graph of the blood vessel diameter changing over time; and
selecting a waveform graph of the arterial blood vessel according to a change rule of blood vessel diameters of arteries and veins within a normal cardiac cycle and the blood flow direction measured by the second ultrasonic transducer, to identify the arterial blood vessel.

Preferably, the measuring a blood vessel diameter of the arterial blood vessel by using the first ultrasonic transducer in the working region specifically includes:
transmitting ultrasonic waves respectively along the first direction by using a plurality of first ultrasonic transducers in the working region;
calculating blood vessel diameters measured by the first ultrasonic transducers at different positions through two spaced echo signals; and
comprehensively calculating the blood vessel diameter of the arterial blood vessel by using the blood vessel diameters measured by the plurality of first ultrasonic transducers.

Preferably, the measuring a blood flow rate of the arterial blood vessel by using the second ultrasonic transducer in the working region specifically includes:
transmitting ultrasonic waves along the second direction by using a plurality of second ultrasonic transducers in the working region, so that incident ultrasonic waves are reflected by flowing blood cells, and a frequency of reflected echo signals changes, thereby forming a Doppler blood flow frequency shift signal;
obtaining a linear relationship between the Doppler blood flow frequency shift signal and velocity components of the blood cells in a propagation direction of the ultrasonic waves; and
performing spectral analysis on the Doppler blood flow frequency shift signal by using the linear relationship, to obtain the blood flow rate and the blood flow direction of the arterial blood vessel.

Preferably, the working region of the flexible ultrasonic transducer array is located directly above the arterial blood vessel, and the working region changes with a relative position between the flexible ultrasonic transducer array and the arterial blood vessel.

Preferably, the automatic blood pressure monitoring method further includes:
displaying the current blood pressure of the user to the user through a display, and displaying a blood pressure change curve to the user based on blood pressure monitoring data within a preset time period.

According to a second aspect of embodiments of the present invention, an ultrasonic wave-based automatic blood pressure monitoring system is provided, including a processor and a memory, where the processor reads a computer program in the memory, and is configured to perform the following operations:
transmitting an ultrasonic wave based on a flexible ultrasonic transducer array arranged on a body surface of a user, and receiving a vascular image of a target region, to identify an arterial blood vessel;
determining a working region of the flexible ultrasonic transducer array based on a position of the arterial blood vessel, where the working region includes a first ultrasonic transducer configured to measure a blood vessel diameter, and a second ultrasonic transducer configured to measure a blood flow rate and a blood flow direction;
measuring a blood vessel diameter of the arterial blood vessel by using the first ultrasonic transducer in the working region;
measuring a blood flow rate and a blood flow direction of the arterial blood vessel by using the second ultrasonic transducer in the working region;
calculating a current blood pressure of the user through a preset algorithm based on the blood vessel diameter and the blood flow rate of the arterial blood vessel, to complete current blood pressure monitoring; and
performing next blood pressure monitoring at a preset interval.

Compared with the prior art, the present invention has the following beneficial effects:
1. An ultrasonic transducer array mounted on a flexible material is used and attached to human skin, a blood vessel can be automatically identified, and a single measurement can be performed based on a setting, or continuous measurements can be performed at different time intervals, thereby achieving a 24-hour blood pressure monitoring effect in a clinical sense. Comfort can be ensured by using the ultrasonic transducer array mounted on the flexible material, and accuracy of blood pressure monitoring can be ensured in a manner of monitoring the blood pressure by using ultrasonic waves.
2. The flexible ultrasonic transducer array includes a plurality of rows of first ultrasonic transducers arranged along a first direction (perpendicular to a skin surface) and a plurality of rows of second ultrasonic transducers arranged along a second direction (inclined to the skin surface). The first ultrasonic transducers are configured to identify a blood vessel type and measure a blood vessel diameter, and the second ultrasonic transducers are configured to measure a blood flow rate, so that a current blood pressure of a user can be obtained through a calculation via a preset algorithm based on the blood vessel diameter and the blood flow rate of an arterial blood vessel, to implement blood pressure monitoring.
3. Because blood vessel data varies among different users, parameter calibration is performed before each to-be-measured subject is measured, so that a blood pressure calculation can be adjusted differently based on different users in a manner of adding a blood pressure calibration, thereby further improving accuracy of the blood pressure monitoring.
4. The blood vessel diameter may be corrected based on a relative direction between the first ultrasonic transducer in a working region and the arterial blood vessel, and the blood flow rate is corrected based on a relative angle between the second ultrasonic transducer in the working region and the arterial blood vessel, so that more accurate data can be obtained through correction, thereby further improving accuracy of the blood pressure monitoring.

### BRIEF DESCRIPTION OF THE DRAWINGS

FIG. 1 is a schematic structural top view of a flexible ultrasonic transducer array according to an embodiment of the present invention;

FIG. 2 is a schematic structural side view of a flexible ultrasonic transducer array in a use state according to an embodiment of the present invention;

FIG. 3 is an overall flowchart of an ultrasonic wave-based automatic blood pressure monitoring method according to a first embodiment of the present invention;

FIG. 4 is a detailed flowchart of an ultrasonic wave-based automatic blood pressure monitoring method according to a first embodiment of the present invention;

FIG. 5 is a schematic diagram of performing blood pressure calibration on a user and completing blood pressure monitoring in a second embodiment of the present invention; and

FIG. 6 is a structural diagram of an ultrasonic wave-based automatic blood pressure monitoring system according to a fourth embodiment of the present invention.

### DETAILED DESCRIPTION

Technical content of the present invention is described in detail below with reference to the accompanying drawings and specific embodiments.

In embodiments of the present invention, an ultrasonic transducer array mounted on a flexible material is used and attached to human skin, a blood vessel can be automatically identified, and a blood pressure can be measured in real time over 24 hours. It may be understood that comfort can be ensured by using the ultrasonic transducer array mounted on the flexible material, and accuracy of blood pressure monitoring can be ensured in a manner of monitoring the blood pressure by using ultrasonic waves.

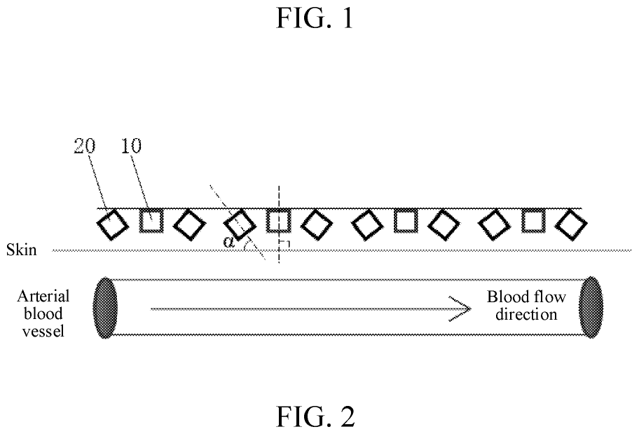

Referring to FIG. 1, in embodiments of the present invention, the flexible ultrasonic transducer array includes a plurality of rows of first ultrasonic transducers 10 (that is, light-colored regions in FIG. 1) arranged along a first direction and a plurality of rows of second ultrasonic transducers 20 (that is, dark-colored regions in FIG. 1) arranged along a second direction. The plurality of rows of first ultrasonic transducers 10 and the plurality of rows of second ultrasonic transducers 20 are arranged parallel to each other, and two rows of second ultrasonic transducers are symmetrically arranged on two sides (that is, upper and lower sides in FIG. 1) of each row of the first ultrasonic transducers. As shown in FIG. 2, when the flexible ultrasonic transducer array is parallel to a skin surface of a user, the first direction is perpendicular to the skin surface of the user (for example, a direction shown by a dashed line in FIG. 2), and the second direction forms an angle α (for example, a direction shown by a dash-dot line in FIG. 2) with the skin surface of the user. Preferably, the angle α ranges from 20° to 60°.

It may be understood that, the first ultrasonic transducers 10 are configured to identify a blood vessel type and measure a blood vessel diameter, and the second ultrasonic transducers 20 are configured to measure a blood flow rate and a blood flow direction, so that current blood pressure of the user may be obtained through a calculation via a preset algorithm based on the blood vessel diameter and the blood flow rate of an arterial blood vessel, to implement blood pressure monitoring.

### First Embodiment

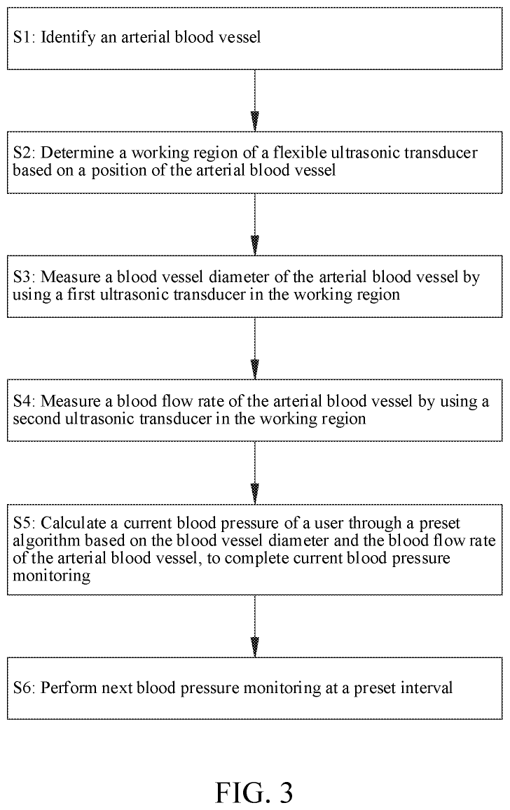

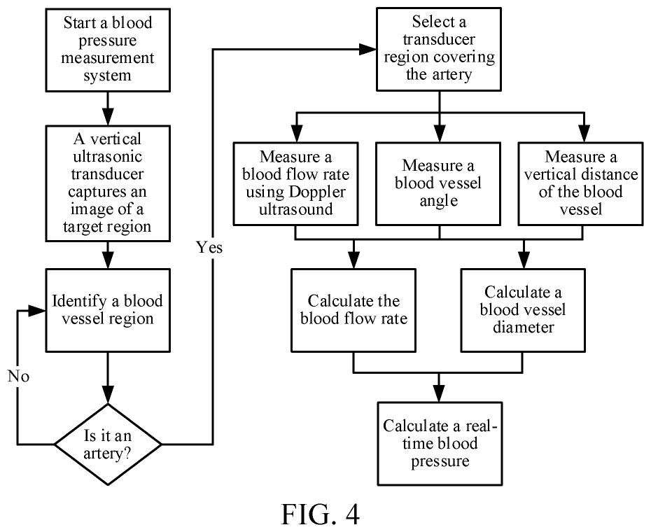

As shown in FIG. 3 and FIG. 4, an ultrasonic wave-based automatic blood pressure monitoring method according to a first embodiment of the present invention specifically includes steps S1 to S6.

S1: Identify an arterial blood vessel.

In this embodiment, an ultrasonic wave is transmitted based on a flexible ultrasonic transducer array arranged on a body surface of a user, and a vascular image of a target region is received, thereby identifying the arterial blood vessel.

Specifically, steps S11 to S13 are included.

S11: Transmit ultrasonic waves toward the target region of the user along a first direction through a plurality of rows of first ultrasonic transducers 10, to calculate a blood vessel diameter along the first direction through two spaced echo signals.

Specifically, the plurality of rows of first ultrasonic transducers 10 transmit and receive the ultrasonic waves along the first direction, to perform imaging processing on the target region. When an ultrasonic signal is transmitted to a target blood vessel, reflection and refraction occur on a front wall and a rear wall of the blood vessel. A diameter of the blood vessel can be obtained by analyzing a time difference between the ultrasonic waves reaching the front wall and the rear wall.

S12: Obtain a waveform graph of the blood vessel diameter changing over time.

It may be understood that, the diameter of the blood vessel may change over time in a cardiac process. The waveform graph of the blood vessel diameter changing over time may be obtained by monitoring the change in the diameter of the target blood vessel.

S13: Select a waveform graph of the arterial blood vessel according to a change rule of blood vessel diameters of arteries and veins within a normal cardiac cycle, to identify the arterial blood vessel.

It may be understood that, because a difference exists in diameter changes between arterial blood vessels and venous blood vessels during a normal cardiac cycle, the arteries and the veins can be successfully distinguished by analyzing the blood vessel diameter changes (that is, observing the waveform graph).

S2: Determine a working region of the flexible ultrasonic transducer array based on a position of the arterial blood vessel.

The working region includes a first ultrasonic transducer configured to measure the blood vessel diameter, and a second ultrasonic transducer configured to measure a blood flow rate.

After the position of the arterial blood vessel is determined based on step S1, an upper region that covers the arterial blood vessel is the working region of the flexible ultrasonic transducer array. In addition, it may be understood that, another ultrasonic transducer in the flexible ultrasonic transducer array other than that in the working region is also in a detection state, while data capturing accuracy of the ultrasonic transducer located in the working region is higher, thereby helping improve accuracy of a subsequent blood pressure calculation.

In addition, when a relative position between the flexible ultrasonic transducer array and the arterial blood vessel changes, the working region changes accordingly, so that data is captured by an ultrasonic transducer at another position.

S3: Measure a blood vessel diameter of the arterial blood vessel by using the first ultrasonic transducer 10 in the working region.

Specifically, steps S31 to S33 are included.

S31: Transmit ultrasonic waves respectively along the first direction by using a plurality of first ultrasonic transducers 10 in the working region.

S32: Calculate blood vessel diameters measured by the first ultrasonic transducers at different positions through two spaced echo signals.

A calculation manner of the blood vessel diameters is the same as the calculation manner of step S11 above. Details are not described herein again. However, it should be understood that step S32 further has the following difference from step S11 above.

The blood vessel diameter measured in step S11 does not need to be very precise, and ultrasonic wave detection in a full coverage manner is performed through all the first ultrasonic transducers 10, to distinguish the arterial blood vessel from numerous blood vessels (only a change trend of the blood vessel diameter needs to be obtained).

However, the blood vessel diameters measured in step S32 are based on having determined the arterial blood vessel, and exact diameters of the arterial blood vessel need to be accurately measured, to be used for the subsequent blood pressure calculation.

S33: Comprehensively calculate the blood vessel diameter of the arterial blood vessel by using the blood vessel diameters measured by the plurality of first ultrasonic transducers.

It may be understood that, the blood vessel diameters measured by the first ultrasonic transducers 10 at different positions may have certain differences, and the first ultrasonic transducers 10 may also have an inherent measurement error. Therefore, the blood vessel diameters measured by the plurality of first ultrasonic transducers 10 are integrated to comprehensively calculate the blood vessel diameter of the arterial blood vessel, so that the measurement error of the blood vessel diameter can be reduced, thereby improving calculation accuracy of the blood pressure.

A comprehensive calculation manner may be determined as needed. For example, an average value of the plurality of blood vessel diameters is obtained, or different weights are assigned to different blood vessel diameters based on blood vessel positions, so that a final blood vessel diameter is calculated based on a weight of each of the blood vessel diameters.

S4: Measure a blood flow rate and a blood flow direction of the arterial blood vessel by using the second ultrasonic transducer 20 in the working region.

Specifically, steps S41 to S43 are included.

S41: Transmit ultrasonic waves along a second direction by using the second ultrasonic transducer 20 in the working region, so that incident ultrasonic waves are reflected by flowing blood cells, and a frequency of reflected echo signals changes, thereby forming a Doppler blood flow frequency shift signal.

S42: Obtain a linear relationship between the Doppler blood flow frequency shift signal and velocity components of the blood cells in a propagation direction of the ultrasonic waves.

S43: Perform spectral analysis on the Doppler blood flow frequency shift signal by using the linear relationship, to obtain the blood flow rate and the blood flow direction of the arterial blood vessel.

It may be understood that, in this embodiment, the second ultrasonic transducers 20 are arranged at an angle of 20° to 60° relative to the skin surface, for the purpose of facilitating the measurement of the blood flow rate using the Doppler frequency shift. In another embodiment, an adaptive adjustment may be performed on a magnitude of the angle based on an actual need.

S5: Calculate a current blood pressure of the user through a preset algorithm based on the blood vessel diameter and the blood flow rate of the arterial blood vessel, to complete current blood pressure monitoring.

The preset algorithm is a built-in machine learning model, and a blood pressure value is continuously calculated through continuous blood vessel diameters and blood flow rates. The changes in the blood vessel diameters that are continuously captured during blood vessel identification may reflect a blood vessel hardening degree and another feature related to the blood pressure through a model calculation. The model is trained using data captured from users in different physical conditions under different scenarios through previous experiments, so that the blood pressure value can be accurately calculated through the captured data.

S6: Perform next blood pressure monitoring at a preset interval.

In the foregoing embodiment, preferably, after blood pressure monitoring is completed once, the current blood pressure of the user may be displayed to the user through a display, and a blood pressure change curve is displayed to the user based on blood pressure monitoring data within a preset time period. Therefore, the user may conveniently view the current blood pressure of the user and blood pressure states in a recent period of time.

Based on the above, according to the ultrasonic wave-based automatic blood pressure monitoring method provided in the first embodiment of the present invention, the ultrasonic transducer array mounted on the flexible material is used and attached to the human skin, so that the arterial blood vessel can be automatically identified, and the blood pressure can be measured in real time over 24 hours, providing not only wearing comfortable but also higher measurement accuracy.

### Second Embodiment

Based on the first embodiment, a second embodiment of the present invention further provides an ultrasonic wave-based automatic blood pressure monitoring method. Compared with the first embodiment, a difference of this embodiment is that before the blood pressure is calculated, blood pressure calibration further needs to be performed on the user a plurality of times. In other words, in this embodiment, step S5 in the first embodiment is replaced with S5′.

S5′: Calculate a current blood pressure of the user, to complete current blood pressure monitoring.

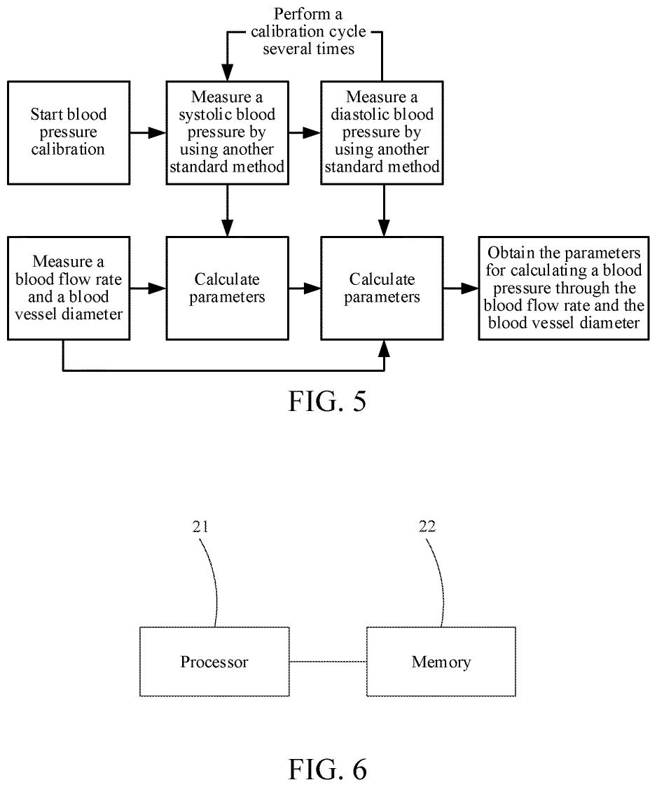

Specifically, as shown in FIG. 5, S51′ to S53′ are included.

S51′: Repeatedly calibrate blood pressures of the user a plurality of times, to obtain calibrated blood pressure values of the user. The calibrated blood pressure values are a systolic blood pressure and a diastolic blood pressure that are measured based on conventional cuff-based blood pressure measurement and another existing standard method.

S52′: Perform parameter fitting on the blood vessel diameter and the blood flow rate of the arterial blood vessel by using the calibrated blood pressure values of the user.

S53′: Calculate the current blood pressure of the user through the preset algorithm based on the blood vessel diameter and the blood flow rate after the parameter fitting.

It may be understood that, because blood vessel data varies among different users, parameter calibration needs to be performed before each to-be-measured subject is measured. After a calibration process is started, the system continuously captures and measures a target blood vessel diameter and a blood flow rate, while simultaneously using the another existing standard method to measure the systolic blood pressure and the diastolic blood pressure, and perform the process several times. Finally, the measurement data and the measured systolic blood pressure and diastolic blood pressure are matched with each other to calculate parameters, which are used for subsequent 24-hour continuous blood pressure monitoring and calculation. Therefore, by adding the blood pressure calibration, the blood pressure calculation can be adjusted differently based on different users, to further improve accuracy of the blood pressure monitoring.

In this embodiment, except for the foregoing steps, the remaining steps are the same as those in the first embodiment. Details are not described herein again.

### Third Embodiment

Based on the first embodiment, a third embodiment of the present invention further provides an ultrasonic wave-based automatic blood pressure monitoring method. Compared with the first embodiment, a difference of this embodiment is that step S5 further includes a data correction process.

Specifically, in this embodiment, step S5 includes steps S51 to S54.

S51: Obtain a relative direction between the first ultrasonic transducer in the working region and the arterial blood vessel based on the position of the arterial blood vessel; and obtain a relative angle between the second ultrasonic transducer in the working region and the arterial blood vessel based on the position of the arterial blood vessel.

It may be understood that, the relative direction refers to that theoretically, an ultrasonic wave transmitting direction of the first ultrasonic transducer should be perpendicular to a length direction of the arterial blood vessel; however, in reality, due to a problem of wearing or mounting accuracy, a certain direction deviation exists between the two, and a magnitude of the direction deviation is the relative direction.

The relative angle refers to that theoretically, an ultrasonic wave transmitting direction of the second ultrasonic transducer should form a certain angle (that is, 20° to 60° as set in this embodiment) with the arterial blood vessel; however, in reality, due to the problem of wearing or mounting accuracy, a certain angular deviation exists between the two, and a magnitude of the angular deviation is the relative angle.

S52: Correct the blood vessel diameter based on the relative direction, to obtain the corrected blood vessel diameter.

S53: Correct the blood flow rate based on the relative angle, to obtain the corrected blood flow rate.

S54: Calculate the current blood pressure of the user through the preset algorithm based on the corrected blood vessel diameter and the corrected blood flow rate.

It may be understood that, data obtained after the correction is more accurate, thereby further improving accuracy of the blood pressure monitoring.

In this embodiment, except for the foregoing steps, the remaining steps are the same as those in the first embodiment. Details are not described herein again.

### Fourth Embodiment

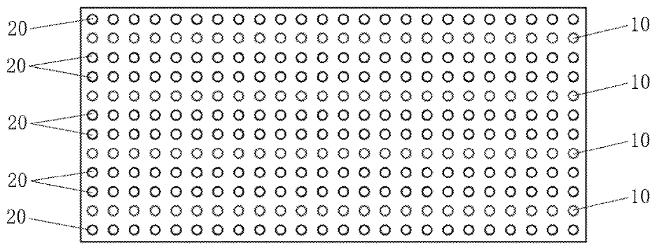

As shown in FIG. 6, based on the foregoing ultrasonic wave-based automatic blood pressure monitoring method, a fourth embodiment of the present invention further provides an ultrasonic wave-based automatic blood pressure monitoring system. The automatic blood pressure monitoring system includes one or more processors 21 and a memory 22. The memory 22 is coupled to the processor 21, and is configured to store one or more programs. The one or more programs, when executed by the one or more processors 21, cause the one or more processors 21 to implement the ultrasonic wave-based automatic blood pressure monitoring method in the foregoing embodiments.

The processor 21 is configured to control an overall operation of the automatic blood pressure monitoring system, to complete all or part of the steps of the foregoing ultrasonic wave-based automatic blood pressure monitoring method. The processor 21 may be a central processing unit (CPU), a graphics processing unit (GPU), a field-programmable gate array (FPGA), an application-specific integrated circuit (ASIC), a digital signal processing (DSP) chip, or the like. The memory 22 is configured to store various types of data to support operations in the automatic blood pressure monitoring system. The data may include, for example, instructions of any application program or method for operation in the automatic blood pressure monitoring system, and data related to the application program. The memory 22 may be implemented by any type of volatile or non-volatile storage device or a combination thereof, for example, a static random access memory (SRAM), an electrically erasable programmable read-only memory (EEPROM), an erasable programmable read-only memory (EPROM), a programmable read-only memory (PROM), a read-only memory (ROM), a magnetic memory, and a flash memory.

In an exemplary embodiment, the automatic blood pressure monitoring system may be specifically implemented by a computer chip or an entity, or implemented by a product having a certain function, and is configured to perform the foregoing ultrasonic wave-based automatic blood pressure monitoring method, and achieve the same technical effect as the foregoing method. A typical embodiment is a computer. Specifically, the computer may be, for example, a personal computer, a laptop computer, an on-board human-computer interaction device, a cellular phone, a camera phone, a smartphone, a personal digital assistant, a media player, a navigation device, an email device, a game console, a tablet computer, a wearable device, or any combination of these devices.

In another exemplary embodiment, the present invention further provides a computer-readable storage medium including program instructions. The program instructions, when executed by a processor, implement the steps of the ultrasonic wave-based automatic blood pressure monitoring method in any one of the foregoing embodiments. For example, the computer-readable storage medium may be the foregoing memory including the program instructions, and the foregoing program instructions may be executed by the processor of the automatic blood pressure monitoring system to complete the foregoing ultrasonic wave-based automatic blood pressure monitoring method, and achieve the same technical effect as the foregoing method.

It should be noted that, the foregoing embodiments are merely examples. The technical solutions of the embodiments may be combined with each other, all of which fall within the protection scope of the present invention.

It should be understood that orientation or position relationships indicated by terms such as “up”, “down”, and “horizontal” are based on orientation or position relationships shown in the accompanying drawings, and are used only for ease of description of the present invention and brevity of the description, rather than indicating or implying that the mentioned apparatus or element needs to have a particular orientation or be constructed and operated in a particular orientation. Therefore, such terms should not be construed as a limitation on the present invention.

In addition, terms “first” and “second” are merely used for the purpose of description, and cannot be construed as indicating or implying relative importance or implicitly indicating a quantity of the indicated technical features. Therefore, a feature defined with “first” and “second” may explicitly or implicitly include one or more of the features. In the descriptions of the present invention, “a plurality of” means two or more, unless otherwise definitely and specifically defined.

The ultrasonic wave-based automatic blood pressure monitoring method and system provided in the present invention are described in detail above. For a person of ordinary skill in the art, any apparent change made to the present invention without departing from the essential content of the present invention constitutes an infringement of the patent right of the present invention, and bears a corresponding legal responsibility.

1-10. (canceled)

11. A flexible ultrasonic transducer array for implementing automated blood pressure monitoring, comprising a plurality of rows of first ultrasonic transducers arranged along a first direction and a plurality of rows of second ultrasonic transducers arranged along a second direction, wherein the plurality of rows of first ultrasonic transducers and the plurality of rows of second ultrasonic transducers are arranged in parallel to each other, and two rows of second ultrasonic transducers are symmetrically arranged on two sides of each row of the first ultrasonic transducers;
when the flexible ultrasonic transducer array is parallel to a skin surface of a user, the first direction is perpendicular to the skin surface of the user, and the second direction is inclined to the skin surface of the user;
wherein the first ultrasonic transducers are configured to measure a blood vessel diameter, and the second ultrasonic transducers are configured to measure a blood flow rate.

12. The flexible ultrasonic transducer array according to claim 11, wherein the first ultrasonic transducers are further configured to identify a blood vessel type, and the second ultrasonic transducers are further configured to measure a blood flow direction.

13. The flexible ultrasonic transducer array according to claim 11, wherein an angle between the second direction and the skin surface of the user ranges from 20° to 60°.

14. An ultrasonic wave based automated blood pressure monitoring method, implemented based on the flexible ultrasonic transducer array according to claim 11, comprising the following steps:
transmitting an ultrasonic wave based on the flexible ultrasonic transducer array arranged on a body surface of a user, and receiving a vascular image of a target region, to identify an arterial blood vessel;
determining a working region of the flexible ultrasonic transducer array based on a position of the arterial blood vessel, wherein the working region comprises a first ultrasonic transducer configured to measure a blood vessel diameter, and a second ultrasonic transducer configured to measure a blood flow rate and a blood flow direction;
measuring a blood vessel diameter of the arterial blood vessel by using the first ultrasonic transducer in the working region;
measuring a blood flow rate and a blood flow direction of the arterial blood vessel by using the second ultrasonic transducer in the working region;
calculating a current blood pressure of the user through a preset algorithm based on the blood vessel diameter and the blood flow rate of the arterial blood vessel, to complete current blood pressure monitoring; and
performing next blood pressure monitoring at a preset interval.

15. The automated blood pressure monitoring method according to claim 14, wherein the calculating a current blood pressure of the user through a preset algorithm based on the blood vessel diameter and the blood flow rate of the arterial blood vessel is replaced with:
repeatedly calibrating blood pressures of the user a plurality of times, to obtain calibrated blood pressure values of the user;
performing parameter fitting on the blood vessel diameter and the blood flow rate of the arterial blood vessel by using the calibrated blood pressure values of the user; and
calculating the current blood pressure of the user through the preset algorithm based on the blood vessel diameter and the blood flow rate after the parameter fitting.

16. The automated blood pressure monitoring method according to claim 14, wherein before the calculating a current blood pressure of the user through a preset algorithm, the method further comprises:
obtaining a relative direction between the first ultrasonic transducer in the working region and the arterial blood vessel based on the position of the arterial blood vessel; and obtaining a relative angle between the second ultrasonic transducer in the working region and the arterial blood vessel based on the position of the arterial blood vessel;
correcting the blood vessel diameter based on the relative direction, to obtain the corrected blood vessel diameter;
correcting the blood flow rate based on the relative angle, to obtain the corrected blood flow rate; and
calculating the current blood pressure of the user through the preset algorithm based on the corrected blood vessel diameter and the corrected blood flow rate.

17. The automated blood pressure monitoring method according to claim 14, wherein the transmitting an ultrasonic wave based on the flexible ultrasonic transducer array arranged on a body surface of a user, and receiving a vascular image of a target region, to identify an arterial blood vessel specifically comprises:
transmitting ultrasonic waves toward the target region of the user along the first direction through the plurality of rows of first ultrasonic transducers, to calculate a blood vessel diameter along the first direction through two spaced echo signals;
obtaining a waveform graph of the blood vessel diameter changing over time; and
selecting a waveform graph of the arterial blood vessel according to a change rule of blood vessel diameters of arteries and veins within a normal cardiac cycle and the blood flow direction measured by the second ultrasonic transducers, to identify the arterial blood vessel.

18. The automated blood pressure monitoring method according to claim 14, wherein the measuring a blood vessel diameter of the arterial blood vessel by using the first ultrasonic transducer in the working region specifically comprises:
transmitting ultrasonic waves respectively along the first direction by using a plurality of first ultrasonic transducers in the working region;
calculating blood vessel diameters measured by the first ultrasonic transducers at different positions through two spaced echo signals; and
comprehensively calculating the blood vessel diameter of the arterial blood vessel by using the blood vessel diameters measured by the plurality of first ultrasonic transducers.

19. The automated blood pressure monitoring method according to claim 14, wherein the measuring a blood flow rate of the arterial blood vessel by using the second ultrasonic transducer in the working region specifically comprises:
transmitting ultrasonic waves along the second direction by using a plurality of second ultrasonic transducers in the working region, so that incident ultrasonic waves are reflected by flowing blood cells, and a frequency of reflected echo signals changes, thereby forming a Doppler blood flow frequency shift signal;
obtaining a linear relationship between the Doppler blood flow frequency shift signal and velocity components of the blood cells in a propagation direction of the ultrasonic waves; and
performing spectral analysis on the Doppler blood flow frequency shift signal by using the linear relationship, to obtain the blood flow rate and the blood flow direction of the arterial blood vessel.

20. The automated blood pressure monitoring method according to claim 14, wherein:
the working region of the flexible ultrasonic transducer array is located directly above the arterial blood vessel, and the working region changes with a relative position between the flexible ultrasonic transducer array and the arterial blood vessel.

21. The automated blood pressure monitoring method according to claim 14, further comprising:
displaying the current blood pressure of the user to the user through a display, and displaying a blood pressure change curve to the user based on blood pressure monitoring data within a preset time period.

22. An ultrasonic wave based automated blood pressure monitoring system, comprising a processor and the flexible ultrasonic transducer array according to claim 11, wherein:
the flexible ultrasonic transducer array is arranged on a body surface of a user, and is configured to transmit an ultrasonic wave and receive a vascular image of a target region;
the processor is configured to identify an arterial blood vessel based on the vascular image;
the processor is further configured to determine a working region of the flexible ultrasonic transducer array based on a position of the arterial blood vessel, wherein the working region comprises a first ultrasonic transducer configured to measure a blood vessel diameter, and a second ultrasonic transducer configured to measure a blood flow rate and a blood flow direction;
the processor is further configured to measure a blood vessel diameter of the arterial blood vessel by using the first ultrasonic transducer in the working region, measure a blood flow rate and a blood flow direction of the arterial blood vessel by using the second ultrasonic transducer in the working region, and calculate a current blood pressure of the user through a preset algorithm based on the blood vessel diameter and the blood flow rate of the arterial blood vessel;
wherein the working region changes with a relative position between the flexible ultrasonic transducer array and the arterial blood vessel.

23. The automated blood pressure monitoring system according to claim 22, wherein the processor is further configured to:
obtain a relative direction between the first ultrasonic transducer in the working region and the arterial blood vessel based on the position of the arterial blood vessel, and correct the blood vessel diameter based on the relative direction;
obtain a relative angle between the second ultrasonic transducer in the working region and the arterial blood vessel based on the position of the arterial blood vessel, and correct the blood flow rate based on the relative angle; and
calculate the current blood pressure of the user based on the corrected blood vessel diameter and the corrected blood flow rate.

24. The automated blood pressure monitoring system according to claim 22, wherein the processor identifying an arterial blood vessel based on the vascular image specifically comprises:
transmitting ultrasonic waves toward the target region of the user along the first direction through the plurality of rows of first ultrasonic transducers, to calculate a blood vessel diameter along the first direction through two spaced echo signals;
obtaining a waveform graph of the blood vessel diameter changing over time; and
selecting a waveform graph of the arterial blood vessel according to a change rule of blood vessel diameters of arteries and veins within a normal cardiac cycle and the blood flow direction measured by the second ultrasonic transducers, to identify the arterial blood vessel.

25. The automated blood pressure monitoring system according to claim 22, wherein the processor measuring a blood vessel diameter of the arterial blood vessel by using the first ultrasonic transducer in the working region specifically comprises:
transmitting ultrasonic waves respectively along the first direction by using a plurality of first ultrasonic transducers in the working region;
calculating blood vessel diameters measured by the first ultrasonic transducers at different positions through two spaced echo signals; and
comprehensively calculating the blood vessel diameter of the arterial blood vessel by using the blood vessel diameters measured by the plurality of first ultrasonic transducers.

26. The automated blood pressure monitoring system according to claim 22, wherein the processor is further configured to:
repeatedly calibrate blood pressures of the user a plurality of times, to obtain calibrated blood pressure values of the user;
perform parameter fitting on the blood vessel diameter and the blood flow rate of the arterial blood vessel by using the calibrated blood pressure values of the user; and
calculate the current blood pressure of the user through the preset algorithm based on the blood vessel diameter and the blood flow rate after the parameter fitting.

27. The automated blood pressure monitoring system according to claim 22, wherein the processor is further configured to:
display the current blood pressure of the user to the user through a display, and display a blood pressure change curve to the user based on blood pressure monitoring data within a preset time period.
